(19) United States
(12) Patent Application Publication
(10) Pub. No.: US 2026/0337543 A1
(43) Pub. Date: Oct. 29, 2026

(54) METHOD AND SYSTEM FOR CELL CULTURE

(71) Applicants: LIFE TECHNOLOGIES CORPORATION, Carlsbad, CA (US); Thermo Scientific Portable Analytical Instruments Inc., Tewksbury, MA (US)

(72) Inventors: Matthew Zustiak, St. Louis, MO (US); Nimesh Khadka, Dracut, MA (US); Juan A. Villa Torrecilla, Madrid (ES); Lin Zhang, Andover, MA (US); David Ramirez Cadavid, St. Louis, MO (US)

(21) Appl. No.: 19/658,142

(22) Filed: Apr. 24, 2026

Related U.S. Application Data
(60) Provisional application No. 63/795,063, filed on Apr. 25, 2025.

(51) Int. Cl. C12M 1/36; C12M 1/00

(52) U.S. Cl. CPC C12M 41/48; C12M 29/00

(57) ABSTRACT

Systems and methods for controlling a bioreactor are disclosed. Spectral data, such as Raman spectral data, is acquired from a cell culture media within a bioreactor and processed using a model system to generate predicted analyte values, including glucose and lactate concentrations. A composite carbon source value is determined by combining at least the predicted glucose value and the predicted lactate value. The carbon source value is compared to a predetermined threshold, and operation of a pump system is controlled to add nutrients to the cell culture media based on the comparison. In some embodiments, the model system evaluates the carbon source value according to one or more selection criteria associated with the model system to assess prediction reliability prior to controlling the pump system. The disclosed systems and methods enable improved representation of the metabolic state of the cell culture and more robust control of bioreactor processes.

### CROSS-REFERENCE TO RELATED APPLICATIONS

This application claims the benefit of and priority to U.S. Provisional Application No. 63/795,063, filed on April 25, 2025, the entirety of which is hereby incorporated by reference herein.

### BACKGROUND

Bioreactors are widely used in the biopharmaceutical industry for the production of biologics, including monoclonal antibodies and other therapeutic proteins. In many implementations, fed-batch bioreactors are employed, wherein nutrients such as glucose are periodically introduced into a cell culture medium to support cell growth and productivity.

In conventional fed-batch processes, the timing and quantity of nutrient additions are typically determined based on periodic sampling and offline analytical measurements. For example, glucose concentration may be measured using laboratory-based assays, and feeding decisions are made according to predefined schedules or threshold-based criteria. These approaches are often labor-intensive and may introduce delays between sampling and control actions. Additionally, reliance on manual sampling and offline analysis can lead to variability in process control and potential inconsistencies in product quality.

To address these limitations, process analytical technologies (PAT), including in-line Raman spectroscopy, have been introduced for real-time monitoring of bioreactor conditions. Raman spectroscopy enables non-invasive, in situ measurement of analytes within a bioreactor, allowing for continuous or near-continuous acquisition of spectral data. Chemometric models may be applied to the acquired spectral data to predict concentrations of analytes such as glucose and lactate in real time.

Prior approaches have demonstrated the use of Raman-based predictions of glucose concentration to automate feeding strategies in bioreactors. For example, predicted glucose values may be used to trigger feeding events when the glucose concentration falls below a predetermined threshold, thereby mimicking conventional fed-batch workflows in an automated manner. While such approaches improve efficiency and reduce reliance on manual sampling, they typically focus on controlling a single analyte and do not fully account for the broader metabolic state of the cell culture.

In particular, lactate is a metabolite that is closely associated with cellular metabolism and can significantly impact cell health, productivity, and product quality. The relationship between glucose consumption and lactate production reflects underlying metabolic dynamics within the bioreactor. However, conventional control strategies generally treat glucose and lactate independently, rather than incorporating both analytes into a unified control framework.

Furthermore, existing process control approaches may not fully leverage the information contained in multiple analytes simultaneously, and may fail to account for interactions between metabolites that influence the overall metabolic state of the cell culture. This can result in suboptimal feeding decisions, particularly in dynamic bioprocess environments where analyte concentrations and metabolic activity change over time.

As a result, there remains a need for improved systems and methods for controlling bioreactors that leverage multi-analyte information to more accurately represent the metabolic state of the culture and to enable more robust and reliable process control.

### BRIEF SUMMARY

The present disclosure generally relates to systems and methods for controlling a bioreactor using spectroscopic data. More particularly, the disclosure relates to utilizing spectral data, such as Raman spectral data, to generate predicted analyte values and to control operation of a bioreactor based on a composite value derived from multiple analytes.

In one aspect, a method for controlling a bioreactor is provided. The method may include acquiring spectral data from a cell culture media disposed within a bioreactor, generating a predicted glucose value and a predicted lactate value from the spectral data, and determining a carbon source value by combining at least the predicted glucose value and the predicted lactate value. The method may further include comparing the carbon source value to a predetermined threshold and controlling operation of a pump system of the bioreactor to add a nutrient to the cell culture media based on the comparison.

In another aspect, a method for controlling a bioreactor is provided in which a carbon source value is generated from spectral data according to a model system. The method may include determining whether the carbon source value satisfies at least one selection criterion associated with the model system, and controlling operation of a pump system of the bioreactor based on the carbon source value and the at least one selection criterion.

In a further aspect, a system for controlling a bioreactor is provided. The system may include a bioreactor having an interior chamber configured to contain a cell culture media and a pump system, a probe configured to acquire spectral data from the cell culture media, and an analyzer configured to receive the spectral data from the probe. The system may further include a control system configured to receive the spectral data from the analyzer, the control system including a model system configured to generate a carbon source value from the spectral data using a glucose model and a lactate model, and a supervisory control component configured to control operation of the pump system based on the carbon source value.

In certain embodiments, the model system may be configured to process spectral data using one or more chemometric or machine learning techniques. The spectral data may be preprocessed using one or more techniques, such as filtering, normalization, and centering, to improve model performance. The model system may further include a validation component configured to evaluate predicted values using one or more selection criteria associated with the model system.

In some embodiments, spectral data and reference data may be associated based on temporal information to facilitate model development. In certain implementations, multiple spectral data points corresponding to a reference measurement may be combined to improve signal quality.

In various embodiments, the use of a composite carbon source value derived from multiple analytes enables improved representation of the metabolic state of the cell culture, thereby allowing for more robust and reliable control of the bioreactor as compared to approaches that rely on a single analyte.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A illustrates a schematic view of an automation system for controlling a bioreactor in accordance with one embodiment.

FIG. 1B illustrates a schematic view of a bioreactor including a pump system and associated components in accordance with one embodiment.

FIG. 1C illustrates a schematic view of an optical system for acquiring spectral data, such as Raman spectral data, in accordance with one embodiment.

FIG. 2A illustrates a schematic view of an analyzer, probe, and control system configured to acquire and transmit spectral data in accordance with one embodiment.

FIG. 2B illustrates a schematic view of a control system including a model system and supervisory control component for generating analyte predictions and controlling operation of a bioreactor in accordance with one embodiment.

FIG. 2C illustrates a schematic view of a computing device associated with the control system in accordance with one embodiment.

FIG. 2D illustrates a flow diagram of a model building method for generating and updating a chemometric model in accordance with one embodiment.

FIG. 2E illustrates a flow diagram of a temporal alignment process for associating spectral data with reference data in accordance with one embodiment.

FIG. 2F illustrates a table and associated data relating to spectral region selection, preprocessing, and model parameters for analyte prediction models in accordance with one embodiment.

FIG. 2G illustrates plots associated with a glucose model, including spectral data, model performance metrics, and feature importance metrics in accordance with one embodiment.

FIG. 2H illustrates plots associated with a lactate model, including spectral data, model performance metrics, and feature importance metrics in accordance with one embodiment.

FIG. 2I illustrates correlation plots between measured and predicted analyte values for glucose and lactate models in accordance with one embodiment.

FIG. 3 illustrates a comparison of control strategies for a bioreactor, including continuous glucose control, standard control, and composite carbon source control in accordance with one embodiment.

FIG. 4 illustrates example plots of analyte concentrations over time for different control strategies in accordance with one embodiment.

FIG. 5 illustrates additional plots of analyte prediction and model performance for different control strategies in accordance with one embodiment.

FIG. 6 illustrates plots of viable cell density and cell viability for different control strategies in accordance with one embodiment.

FIG. 7A illustrates a plot of predicted glucose concentration over time for different control strategies in accordance with one embodiment.

FIG. 7B illustrates a plot of measured lactate concentration over time for different control strategies in accordance with one embodiment.

FIG. 7C illustrates a plot of product titer over time for different control strategies in accordance with one embodiment.

FIG. 8A illustrates a plot of predicted carbon source values over time for different control strategies in accordance with one embodiment.

FIG. 8B illustrates a plot of measured carbon source values over time for different control strategies in accordance with one embodiment.

FIG. 9 illustrates plots of product quality metrics, including size-exclusion chromatography (SEC) and glycation, for different control strategies in accordance with one embodiment.

FIG. 10 illustrates a flow diagram of a control routine for adjusting a bioreactor based on predicted analyte values in accordance with one embodiment.

FIG. 11 illustrates a detailed flow diagram of a control routine including acquiring spectral data, predicting analyte values, calculating a carbon source value, comparing to a threshold, and adjusting the bioreactor in accordance with one embodiment.

### DETAILED DESCRIPTION

Before describing the present disclosure in detail, it is to be understood that this disclosure is not limited to particularly exemplified apparatus, systems, methods, or process parameters that may, of course, vary. It is also to be understood that the terminology used herein is only for the purpose of describing particular embodiments of the present invention, and is not intended to limit the scope of the invention in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” “having” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “port” includes one, two, or more ports.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the invention or claims.

Where possible, like numbering of elements have been used in various figures. Furthermore, multiple instances of an element and or sub-elements of a parent element may each include separate letters appended to the element number. For example two instances of a particular element “91” may be labeled as “91a” and “91b”. In that case, the element label may be used without an appended letter (e.g., “91”) to generally refer to instances of the element or any one of the elements. Element labels including an appended letter (e.g., “91a”) can be used to refer to a specific instance of the element or to distinguish or draw attention to multiple uses of the element. Furthermore, an element label with an appended letter can be used to designate an alternative design, structure, function, implementation, and/or embodiment of an element or feature without an appended letter. Likewise, an element label with an appended letter can be used to indicate a sub-element of a parent element. For instance, an element “12” can comprise sub-elements “12a” and “12b.”

Various aspects of the present devices and systems may be illustrated by describing components that are coupled, attached, and/or joined together. As used herein, the terms “coupled”, “attached”, “connected” and/or “joined” are used to indicate either a direct connection between two components or, where appropriate, an indirect connection to one another through intervening or intermediate components. In contrast, when a component is referred to as being “directly coupled”, “directly attached”, “directly connected” and/or “directly joined” to another component, there are no intervening elements present.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

Various embodiments of the present disclosure generally relate to an automation system 100 generally configured to control one or more processes of a bioreactor 102 during the performance of a bioprocess. More specifically, such an automation system 100 may be configured to control automated feed strategies for a cell culture media 116 disposed within the bioreactor 102 according to real-time monitoring of the conditions thereof. Particularly, the automation system 100 of various embodiments of the present disclosure may be configured to track the glucose consumption and lactate production and, in response thereto, control a pump system 110 of the bioreactor 102 to maintain the overall metabolic state of the cell culture media 116 at an optimal condition for the target analyte.

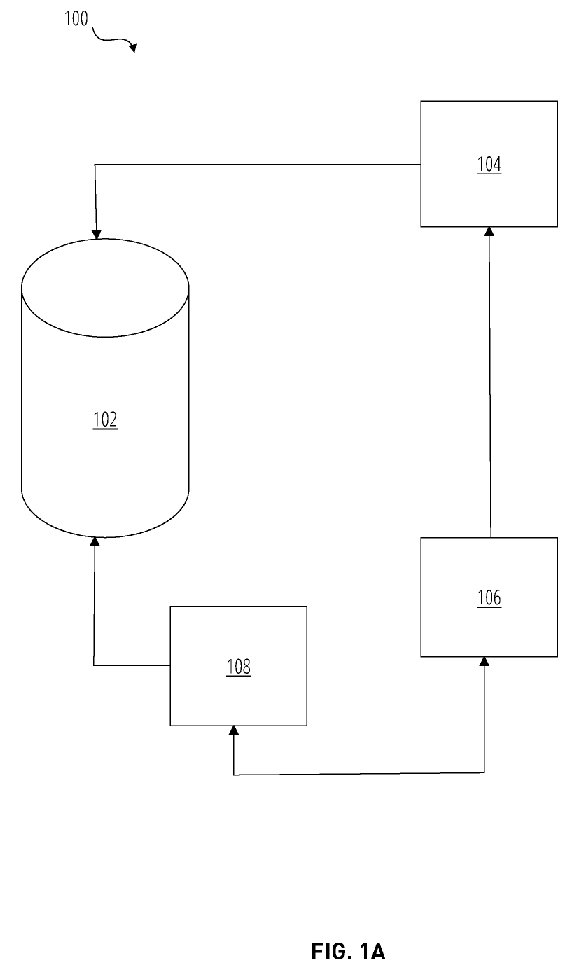

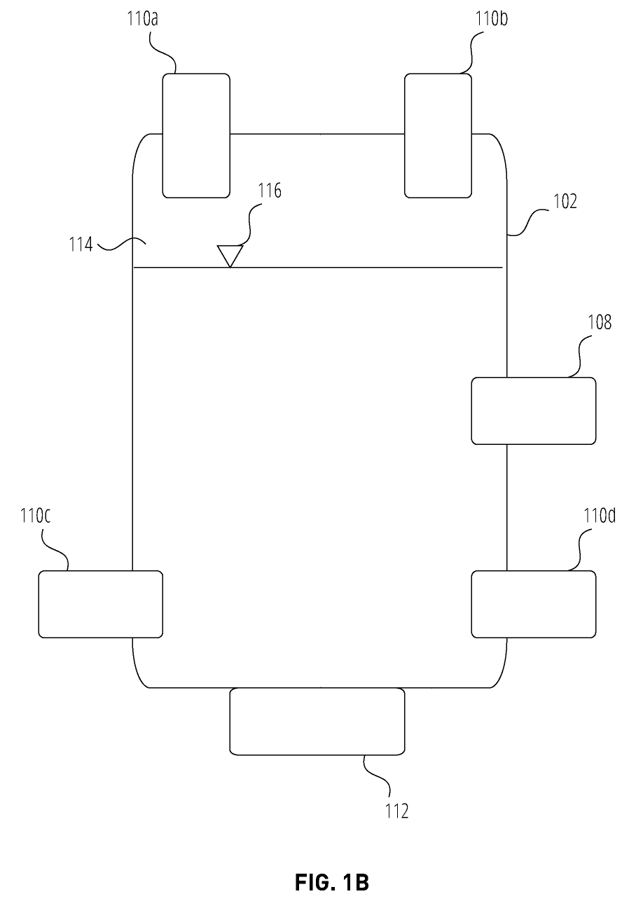

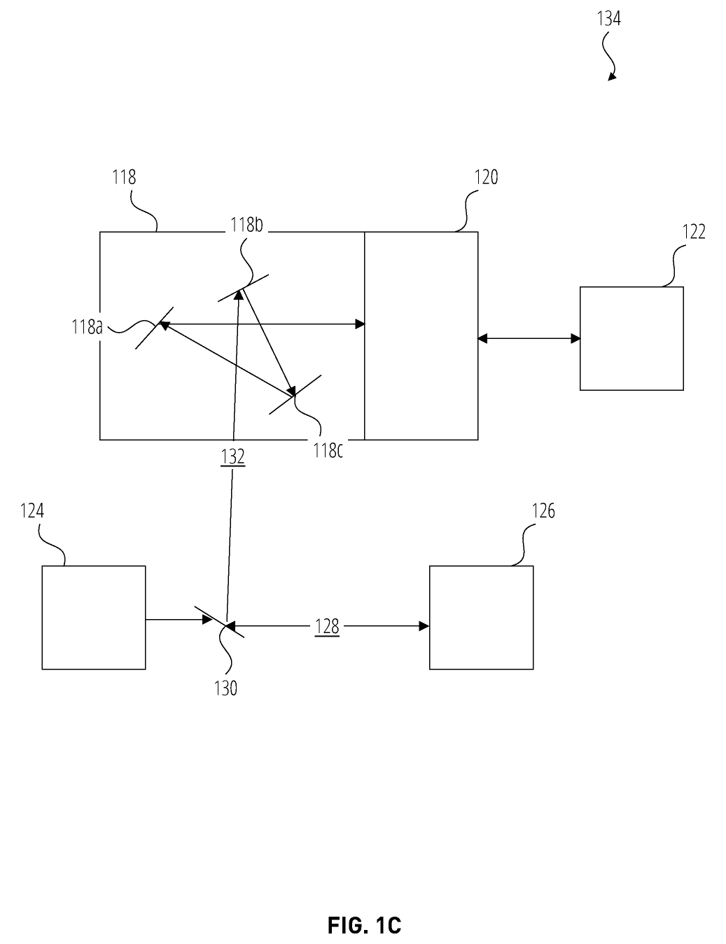

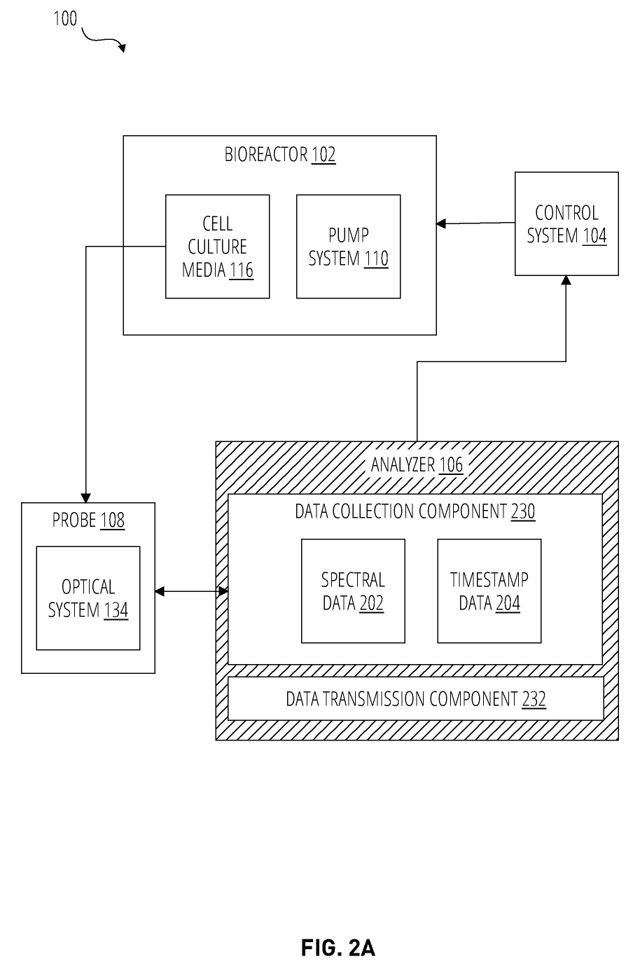

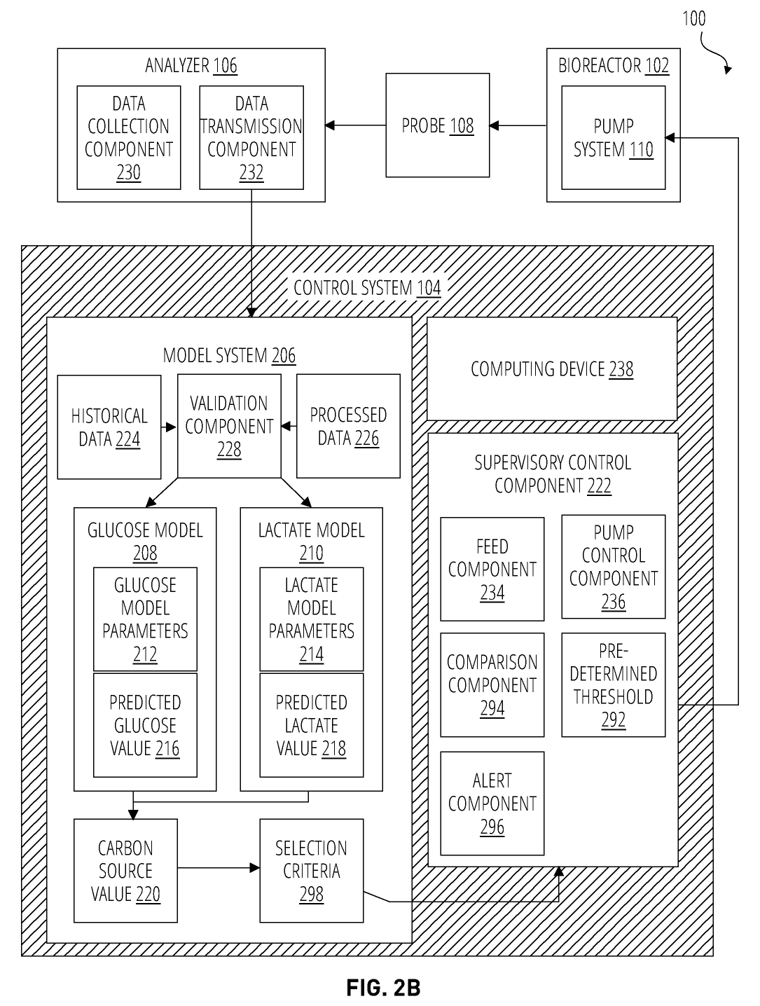

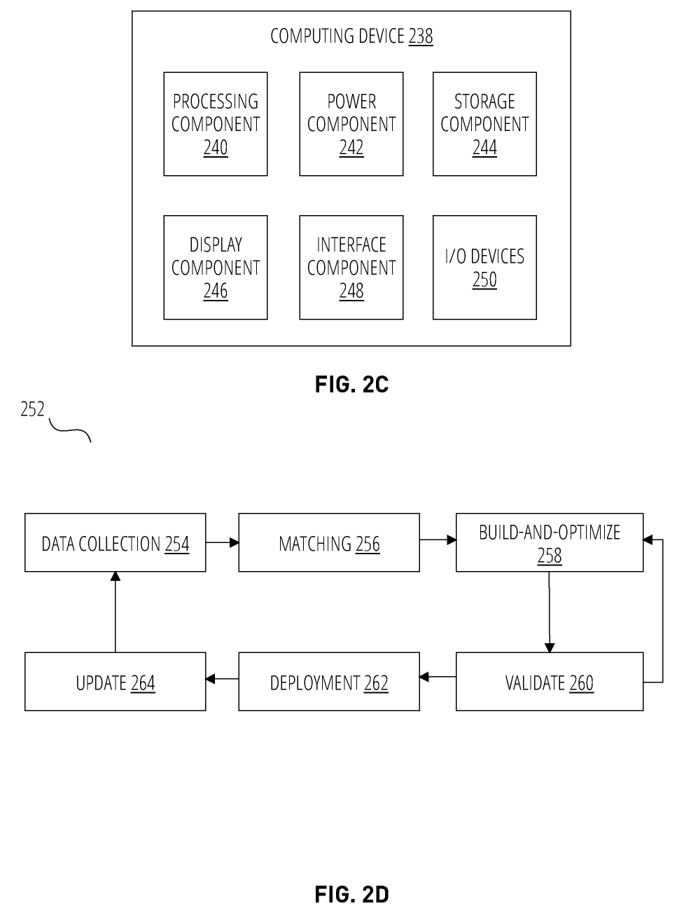

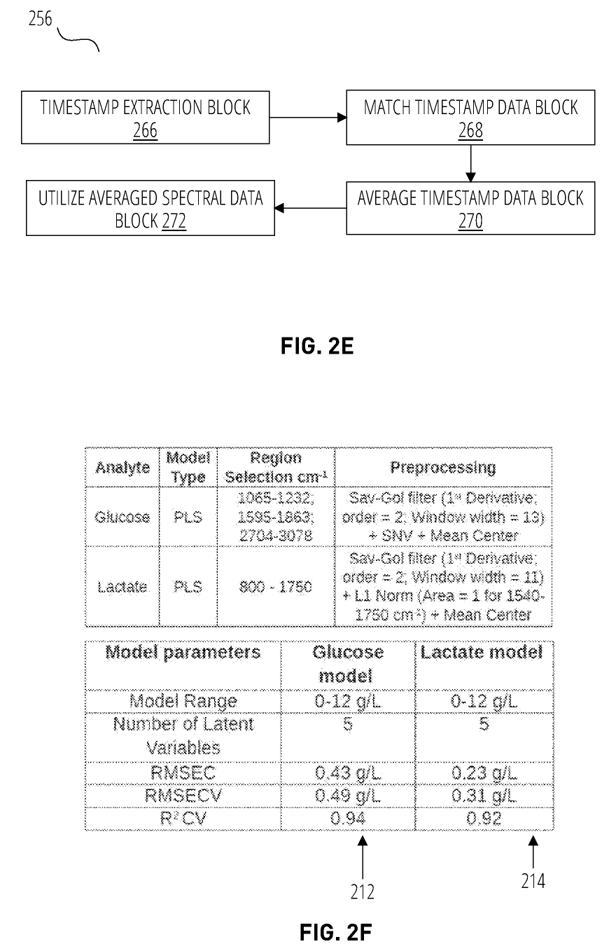

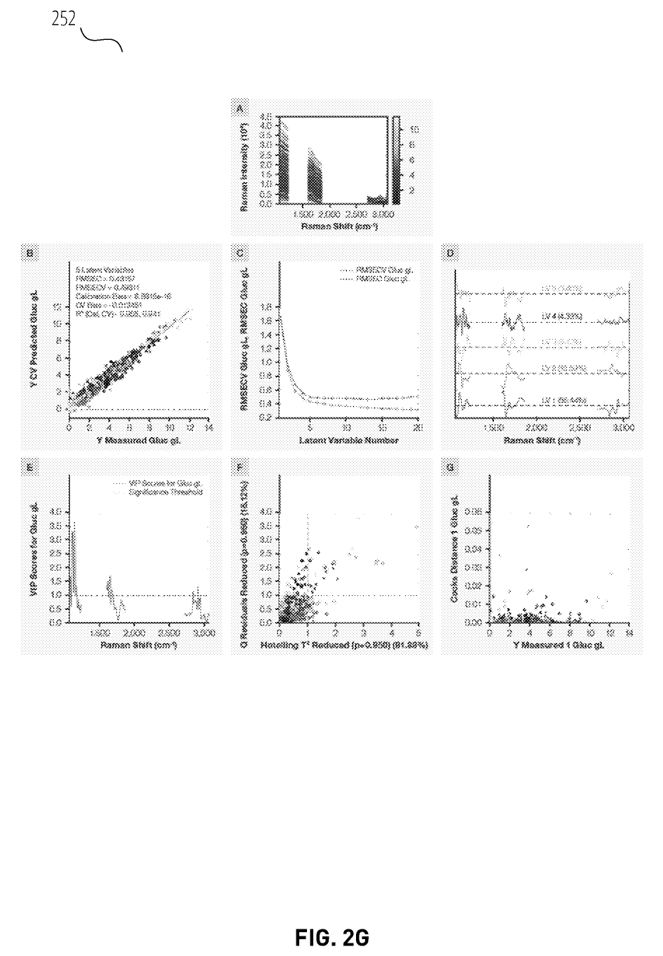

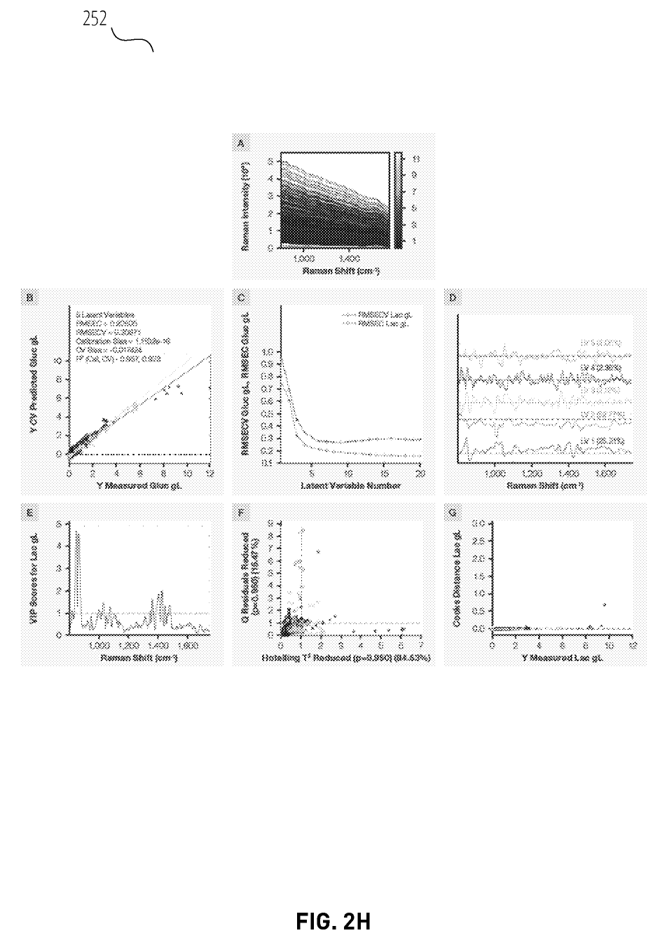

For instance, various embodiments of such an automation system 100 may be seen with reference to FIGS. 1A–2C. In at least one embodiment, such an automation system 100 may include a bioreactor 102, a control system 104, and an analyzer 106 communicatively coupled to a probe 108.

Such a bioreactor 102 may include an interior chamber 114 configured to contain a cell culture media 116. Such a bioreactor 102 may further include a pump system 110 comprising one or more pumps, such as a feed pump 110a, a glucose pump 110b, a recirculatory pump 110c, and a bleed pump 110d, each of which may be configured to introduce or remove materials from the cell culture media 116. Such a bioreactor 102 may further be configured to enable one or more probes 108 to enter into the interior chamber 114, whether tangibly or otherwise, for interaction with the cell culture media 116. In at least one embodiment, such a bioreactor 102 may further include a weighing component 112 configured to track the weight of the cell culture media 116 disposed therein.

As used herein, such a bioreactor 102 is a device configured to retain such a cell culture media 116 whilst maintaining a favorable environment for cell growth, and may generally comprise a bioreactor, a stirred-tank reactor, an adherent bioreactor, a wave-type bioreactor, a disposable bioreactor, a fermenter, and other similar such bioprocessing vessels as understood by those having skill in the art. Such a bioreactor 102 may be configured for use with a variety of different cell lines and different initial and feed media, and may be configured to operate in various modes, including fed-batch, perfusion, and hybrid perfusion, wherein a variety of feeding strategies, such as bolus, continuous, or some combination thereof, may be employed therewith. In at least one embodiment, such a bioreactor 102 may comprise, without limitation, a 3 L glass bioreactor, a 5 liter glass bioreactor, and/or a 500 L single use bioreactor, such as the DynaDrive^{TM} single-use bioreactor (S.U.B.) offered by Thermo Scientific^{TM}.

Such an analyzer 106 may be configured to acquire spectral data 202 from the cell culture media 116 through the probe 108. In various embodiments, such a probe 108 may comprise a Raman probe having an optical system 134 configured to direct excitation light into the cell culture and collect scattered light for subsequent analysis. Such an analyzer 106 may include a data collection component 230 configured to receive the spectral data 202 from the probe 108, and a data transmission component 232 configured to transmit the spectral data 202 to a control system 104 communicatively configured in connection therewith. In at least one embodiment herein, such an analyzer 106 and probe 108 may comprise the Thermo Scientific^{TM} MarqMetrix^{TM} All-in-One Process Raman Analyzer integrated with an immersible probe, although other similar such Raman analyzers and/or probes are contemplated herein.

For instance, the optical system 134 of the probe 108 for acquiring the Raman spectral data 202 of the cell culture media 116 of at least one embodiment of the present disclosure may be seen with reference to FIG. 1C. As may be seen, such an optical system 134 may be configured to directionally control beam path 128 and/or beam path 132. In certain embodiments, such beam path 128 and/or beam path 132 may be controlled using one or more turning mirrors, waveguide phase scrambles, various lenses, broadband filters, or selective elements (e.g., mirrors, notch filters, or other elements with substantially reflective characteristics relative to the wavelength(s) of the beam generated by the Raman laser 124, and/or substantially transmissive characteristics to a wavelength, or desired wavelength, associated with the Raman scattered light from sample 126). In the presently described example, selective element 130 is transmissive to the laser wavelength emitted from Raman laser 124, thereby enabling the beam path 128 to be focused on the sample 126, such as through one or more lenses. Such a lens may include any type of lens known in the art, such as an objective lens or lens architecture configured to focus the beam path 128 onto the sample 126.

In certain embodiments, such a lens may include certain configurations and characteristics that provides advantages for different types of samples. For example, lens may be configured to collect Raman scattered light and Rayleigh scattered light produced from the sample 126 in response to the beam from Raman laser 124. The scattered light collected by the lens may be directed back from the surface of the sample 126 and travel back along the beam path 128 to the selective element 130, which may itself comprise a beam splitter, such as a dichroic mirror, to direct the scattered light along beam path 132. In some implementations, such a selective element 130 is substantially reflective to the wavelengths of the Raman scattered light, allowing beam path 132 to be directed to additional optical elements that further adjust the path and conditions the characteristics of the beam traveling along beam path 132. As may be understood, alternative optical arrangements are also contemplated herein for the selective element 130 for directing the scattered light along the beam path 132.

In various embodiments herein, the optical components 118 may include one or more optical component 118a-118c, which themselves may include one or more of collimating lens and mirrors, filters, such as a notch filter, diffraction gratings, and/or mirror relays. The scattered light may be directed onto such optical components 118 and onto a detector 120. Signal processing and/or digitizing of signals associated with the scattered light that is received by the detector 120 can then be handled by an electrical signal processor 122 associated with optical system 134. In various embodiments, the electrical signal processor 122 may be a suitable programmed microprocessor or application specific integrated circuit including a read-only or read-write memory of any known type which holds instructions and data for spectrometers operation as described herein. Computer readable instructions may be stored in the memory thereof, and the electrical signal processor 122, or alternatively the control system 104 discussed herein, may be configured to implement the methods disclosed herein.

With reference to FIG. 2B, it may be seen the control system 104 of various embodiments of the present disclosure may generally include a computing device 238, a model system 206, and supervisory control component 222. Each of these devices, systems, and/or components will be discussed in turn hereafter.

At least one embodiment of such a computing device 238 may be seen with reference to FIG. 2C. As may be understood, such a computing device 238 may generally comprise some device, such as a smart phone, laptop, computer, tablet, or other similar device through which a user may interact with the control system 104, and may include a plurality of electrical and electronic components configured to provide power, operational control, and/or protection to the same. For instance, such a computing device 238 may comprise a processing component 240, such as an electronic microprocessor, microcontroller, or other similar device, a power component 242, such as a battery, and a storage component 244, such as a memory, including, without limitation, a non-transitory, computer-readable memory. Likewise, such a computing device 238 may additionally comprise a display component 246, such as a screen or touchscreen, and an interface component 248, which may be configured to receive data and provide data to the user, such as in audible, textual, and/or graphical form. Finally, such a computing device 238 may additionally comprise I/O Devices 250, such as a touchscreen, keypad, keyboard, cursor-controlled device, or other similar component configured to provide input-output functionality. As may be understood, such a computing device 238 may be integrally formed with the control system 104 and/or may be external thereto and communicatively coupled in connection therewith. For instance, in at least one embodiment, one such computing device 238 may be formed as part of the bioreactor 102 itself. As such, it may be understood the control system 104, or certain elements, components, and/or systems thereof, may be configured such that it may be employed on a variety of computing devices 238, such as through an application programming interface, and may be configured in communication with one or more servers and/or other computing devices 238.

Returning to FIG. 2B, the supervisory control component 222 of the control system 104 may generally comprise a supervisory control and data acquisition system for the monitoring and control of the processes of the bioreactor 102. For instance, such a supervisory control component 222 may include a pump control component 236 configured to operate the pump system 110 of the bioreactor 102, such as by opening the various pumps of the pump system 110. In at least one embodiment, such pump control component 236 is configured to operate the pump system 110 in a binary, or ON/OFF manner.

Similarly, such a supervisory control component 222 may comprise an alert component 296 configured to issue one or more alerts, such as alarms, notifications, and the like, to the bioreactor 102 and/or the computing device(s) 238. Further, such a supervisory control component 222 may additionally include specific components attuned to the real-time data collection contemplated herein, such as a feed component 234, a comparison component 294, and a predetermined threshold 292, as will be discussed in greater detail hereafter.

In at least one embodiment of the present disclosure, such a supervisory control component 222 may comprise the TruBio^{TM} 6 software offered by Thermo Fisher^{TM}, which may itself be interconnected with some other control automation platform, such as the DeltaV^{TM} distributed control system offered by Emerson Electric^{TM}. Notwithstanding, alternative SCADA and/or distributed control systems for performing the various functions of the supervisory control component 222 are contemplated herein, as understood by those having skill in the art.

With continued reference to FIG. 2B, it may be seen the control system 104 of at least one embodiment of the present disclosure may additionally include a model system 206. Such a model system 206 may generally comprise one or more chemometric models configured to translate the spectral data 202 acquired by the probe 108 and transmitted by the analyzer 106 into meaningful and actionable information.

Accordingly, it may be seen the model system 206 of various embodiments of the present disclosure may include a glucose model 208 and a lactate model 210, each of which are configured to process the spectral data 202 and generate corresponding predicted analyte values. For instance, the glucose model 208 may generate a predicted glucose value 216, whereas the lactate model 210 may generate a predicted lactate value 218. In various embodiments, such glucose model 208 and such lactate model 210 may be implemented using chemometric techniques, such as partial least squares (PLS) regression, or other machine learning approaches, as described herein. In at least one embodiment, the glucose model 208 and/or the lactate model 210 were developed using Python and Eigenvector SOLO software through an iterative process repeated for all target analytes.

For instance, the glucose model 208 and/or the lactate model 210 of the present disclosure may have been developed according to the process depicted in FIG. 2D, according to a training dataset that may comprise, for instance, historical data 224 and processed data 226. Such historical data 224 may comprise, for instance, a large dataset including associated offline reference values for a variety of target analytes and may, in certain embodiments, be configured to receive additional data from later runs utilizing the glucose model 208 and/or lactate model 210. For instance, the historical data 224 of at least one embodiment of the present disclosure comprised spectral data 202 from 35 bioreactor runs, covering various cell lines, media, processes, scales, instruments, and feeding strategies, such as bolus, continuous, or some combination thereof. Such spectral data 202 was collected at 450 mW power and 3000 ms exposure, averaging 20 acquisitions per spectrum. As may be understood, this process of data collection corresponds to data collection block 254, during which time inline Raman spectral data 202 and offline reference values were collected by the model system 206. In certain embodiments, the historical data 224 for the glucose model 208 and the lactate model 210 were parameterized according to glucose model parameters 212 and lactate model parameters 214 determined during the model training process, as depicted in FIG. 2F.

In connection therewith, training of the glucose model 208 and/or the lactate model 210 additionally involved processed data 226, which was generated according to the historical data 224. For instance, the processed data 226 of at least one embodiment comprises the spectral data 202 and corresponding reference values that were collectively preprocessed according to chemometric calibration techniques. Such preprocessing to generate the processed data 226, as depicted in FIG. 2F, may include the application of filtering, normalization, and centering operations to improve signal quality and model performance.

For example, in certain embodiments, preprocessing may include applying a Savitzky-Golay filter to remove baseline variations and enhance spectral features, followed by normalization techniques such as standard normal variate (SNV) to correct for scattering and intensity variations, and mean-centering to prepare the data for regression modeling. Such preprocessing steps enable improved specificity and robustness of the models, particularly in complex biological environments where multiple spectral contributors may be present.

Additionally, spectral region selection may be performed to focus the model on wavelength regions associated with the target analytes. Likewise, regions corresponding to characteristic vibrational modes of glucose and lactate may be selected to improve model specificity, while other regions may be included to account for background or normalization effects. Collectively, the spectral region selection and preprocessing improved model performance and transferability.

In at least one embodiment, cosmic ray correction was applied to enhance SNR, and five spectra were averaged before model development. To prevent overfitting, LOOCV was used, leaving out one bioreactor dataset per iteration. The optimal number of latent variables was chosen to minimize RMSEC and RMSECV, keeping their ratio close to 1.

With continued reference to FIGS. 2B and 2B, the development of the glucose model 208 and/or lactate model 210 additionally included matching block 256, during which offline reference values were matched with the spectral data 202. In at least one embodiment, the spectral data 202 was acquired at a plurality of time points. And, in certain implementations, that spectral data 202 was associated with the reference values based on temporal information, such as the timestamp data 204 depicted in FIG. 2A, to facilitate alignment between measured and predicted values by the model system 206.

In certain non-limiting embodiments, and without requiring such techniques for operation of the model system 206, temporal alignment of spectral data 202 and reference data may be performed in matching block 256. For instance, depicted in FIG. 2E is one exemplary embodiment of such a temporal-based scheme utilized in matching block 256. Here, in timestamp extraction block 266, the timestamp data 204 is extracted from the metadata associated with each spectral data 202. Then, in match timestamp data block 268, the timestamp data for the spectral data 202 is matched with the timestamp data 204 for the reference values of the historical data 224 for the target analytes. Subsequently, to improve the signal-to-noise ratio (SNR) in average timestamp data block 270, the timestamp data 204 for the spectral data 202 that is centered around the matching timestamp data 204 for the reference values is selected and averaged. In at least one embodiment, five Raman spectral data were selected, providing an averaged Raman spectrum having a total exposure time of around 5 minutes. And, finally, in utilize averaged spectral data block 272, the spectral data 202 associated with the averaged timestamp data 204 matched with the reference values is selected and utilized for the chemometric modeling process. In so doing, the matching block 256 of at least one embodiment of the present disclosure allows for the non-stop, frequent, or continuous collection of Raman spectral data 202 by enabling the precise matching of spectral data 202 to fit the model system 206. As may be understood, and as discussed in greater detail herein, such a matching block 256 may be utilized in connection with not just chemometric models for glucose and lactate, but alternative carbon sources as well.

With continued reference to FIGS. 2B and 2D, additional process steps of the model building method 252 include build-and-optimize block 258 and validate block 260, during which time the glucose model 208 and/or lactate model 210 is iteratively built, validated, and optimized according to the above-referenced processes. In at least one embodiment, validation of the model(s) under validate block 260 occurred through validation component 228, through which a leave-one-out cross-validation (LOOCV) strategy was utilized. Initially, each model was built using different numbers of latent variables (LVs) ranging from 1 to 20. The root mean square error of calibration and cross-validation (RMSEC and RMSECV) were calculated for each model with different numbers of latent variables. The PLS model with the optimum number of latent variables was selected such that, one, adding more latent variables did not significantly improve the RMSECV; and (two) the ratio of RMSEC and RMSECV was close to 1. Subsequent thereto, the glucose model 208 and/or lactate model 210 model was deployed under deployment block 262 and, subsequent to deployment and in connection with the collection of additional historical data 224, such model(s) may be updated under update block 264.

Additional information relating to the model building method 252 of the glucose model 208 and lactate model 210 may be seen with reference to FIGS. 2G and 2H. Specifically, FIG. 2G depicts the glucose model 208 and FIG. 2H depicts the lactate model 210, wherein for each such model: (a) plot A shows the region used to develop the model; (b) plot B shows the correlation between the measured and the prediction during cross validation; (c) plot C shows RMSEC and RMSECV vs. Number of latent variables; (d) plot D shows loading with percent variance captured; (e) plot E shows VIP scores; (f) plot F shows the reduced Q residual vs. Reduced Hotelling T² plot; and (g) plot G is the Cook's distance for all training samples.

Returning now to FIG. 2B, it may be understood the glucose model 208 of various embodiments of the present disclosure may be configured to generate a predicted glucose value 216 according to the model itself, as well as the spectral data 202 collected from the probe 108. Likewise, the lactate model 210 may be similarly configured to generate a predicted lactate value 218 according to the model itself, as well as the spectral data 202 collected from the probe. In at least one embodiment, the glucose model 208 and/or the lactate model 210 may be constructed using partial least squares (PLS), partial least squares regression (PLS1), artificial neural networks (ANN), convolutional neural networks (CNN), support vector regression (SVR), principal component regression (PCR), classification (CLS), and Lighthill-Whitham-Richards modelling (LWR).

Subsequent to the generation of the predicted glucose value 216 and the predicted lactate value 218, the model system 206 may generate a carbon source value 220 via a carbon source value component. As used herein, the carbon energy source, or carbon source in the cell culture media in the cell culture, may include one or more components of glucose, glutamine, pyruvate, lactate, and galactose. The carbon energy source is supplemented in the cell culture media to ensure optimal growth and maintenance of the cells. The value of the carbon energy source represents the amount of the carbon energy source in the cell culture media. The carbon energy source may include the concentration of the carbon energy source or the amount of carbon energy source. In one example, the value of the carbon energy source is the concentration of a majority of the carbon energy sources in the cell culture media. Consequently, the carbon source value 220 represents a composite variable derived from multiple analytes and may be used to characterize a metabolic state of the cell culture within the bioreactor

In some embodiments, the carbon source value 220 is determined by combining at least the predicted glucose value 216 and the predicted lactate value 218. For example, the carbon source value may be calculated as a sum of the predicted analyte values. In other embodiments, the carbon source value may be determined using a weighted combination, nonlinear function, or other multi-variable relationship that reflects contributions of multiple carbon-related analytes.

The use of a composite carbon source value 220 allows the control system 104 to incorporate information from multiple analytes into a single control variable, thereby providing a more comprehensive representation of the metabolic condition of the cell culture than approaches that rely on a single analyte.

As may be noted via continued reference to FIG. 2B, the carbon source value 220 may be provided to the supervisory control component 222, which includes a comparison component 294 configured to compare the carbon source value with a predetermined threshold 292. The predetermined threshold 292 may correspond to a desired concentration or range of carbon availability within the bioreactor. The threshold concentration may be experientially determined. For example, the concentration threshold may be determined for a cell line by culturing the cell line based on feedback control of multiple thresholds. The concentration threshold may be the optimal threshold that resulted in the best performance. The performance may be evaluated based on one or more of the cell viability, cell density, lactate concentration, glucose concentration, titer concentration, SEC analysis, and glycation analysis.

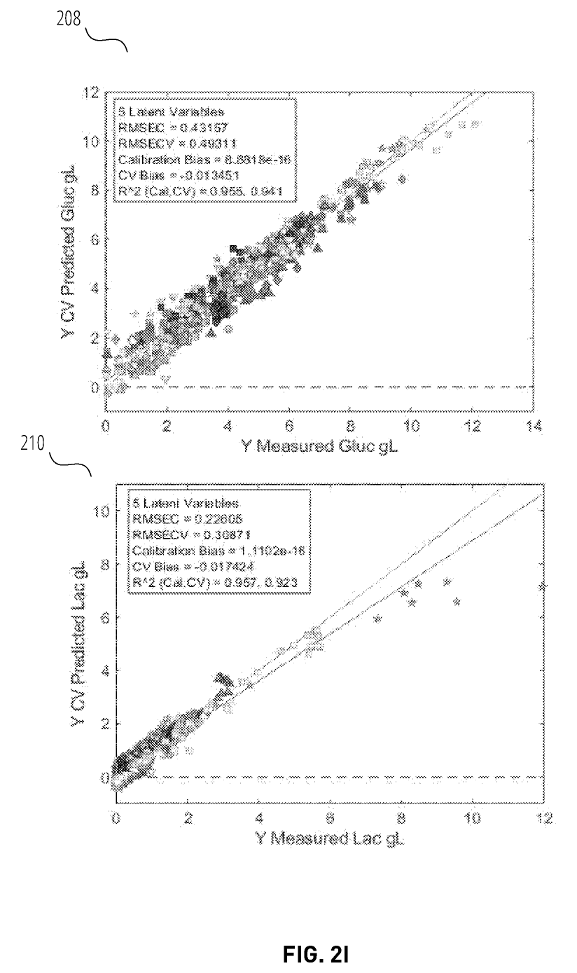

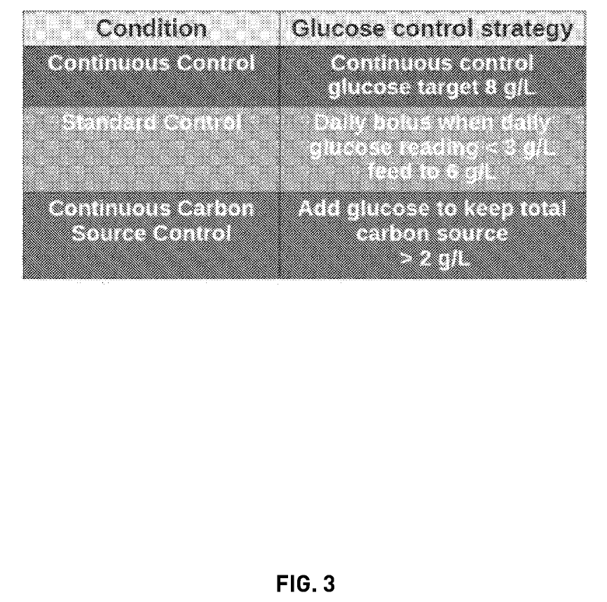

In one example, such as the embodiment depicted in FIG. 3, the predetermined threshold 292 of the carbon source value 220 may be less than 8g/L, less than 3 g/L, or within a range of 1-3g/L, including approximately 2g/L in certain embodiments.

Based on the comparison, the supervisory control component 222 may generate control signals to a pump control component 236, which in turn controls operation of the pump system 110 of the bioreactor. For example, when the carbon source value 220 falls below the predetermined threshold 292, the pump control component 236 may activate a feed component 234 to introduce glucose or other nutrients into the cell culture media. Conversely, when the carbon source value 220 meets or exceeds the threshold, the pump system 110 may be deactivated.

In some embodiments, the control logic may be implemented as a binary control scheme, wherein the pump system 110 is activated or deactivated based on the comparison. In other embodiments, additional control strategies may be employed, such as maintaining the carbon source value 220 within a specified range or executing periodic or bolus feeding operations consistent with fed-batch workflows. Accordingly, it may understood such control strategies may be implemented to automate conventional fed-batch processes, replacing manual sampling and offline analysis with real-time feedback based on spectroscopic measurements.

Likewise, in some embodiments, adjusting the cell culture may include adjusting an amount of one or more components of the carbon energy carbon source value 220 in the cell culture media 116 so that the carbon energy carbon source value 220 reaches to the predetermined threshold 292. For example, responsive to the concentration of the carbon source value 220 concentration lower than the predetermined threshold 292 concentration, a component of the carbon energy source, such as glucose, may be flowed into the bioreactor. The carbon energy source concentration is monitored over time, and substantially in real-time, based on the spectral data acquired from the cell culture, for example, at an interval of 2 minutes. The flow of the component of the carbon energy source to the cell culture media 116 is stopped when the carbon source value 220 concentration reaches the predetermined threshold 292 level. The flow rate of the one or more components of the carbon energy source to the cell culture media 116 may depend on process conditions, including cell density, culture volume, or other operating parameters. For example, the flow rate may depend on the volume of the cell culture and the cell density. The flow rate may further depend on the period at which the spectral data are acquired.

Notwithstanding, it may be seen that, before provision to the supervisory control component 222, the model system 206 may assess the carbon source value 220 in relation to one or more selection criteria 298. This process may be utilized to evaluate the reliability of predictions generated by the glucose model 208 and lactate model 210. Thus, by applying one or more selection criteria 298 associated with glucose model 208 and lactate model 210, the model system 206 may determine whether the predicted glucose value 216 and/or predicted lactate value 218, and by extension the carbon source value 220, satisfies the specified conditions which may be considered by the control system 104 in determining operation of the pump system 110 of the bioreactor 102.

The selection criteria may be based on characteristics of the model system, including feature importance metrics, model performance metrics, or statistical measures associated with the model. For example, metrics such as variable importance in projection (VIP) scores, selectivity ratios, residual analysis, and statistical confidence measures may be used to evaluate model performance and identify potential outliers. If the predicted values do not satisfy the selection criteria 298, the validation component 228 may trigger an alert via the alert component 296 or may otherwise inhibit or modify control actions of the pump system 110. In this manner, the control system may incorporate model validation considerations into the decision-making process, thereby improving robustness and reliability.

For instance, in at least one embodiment of the model system 206 of the present disclosure, the specificity of the glucose model 208 and the lactate model 210 were confirmed using VIP scores and selectivity ratios, identifying characteristic Raman peaks at~ 1125 cm-1 for glucose and ~820 to 880 cm-1 for lactate. As shown in FIG. 2I, the glucose model 208 showed high accuracy and precision with an RMSECV of 0.49 g/L, CV bias of -0.013 g/L, and precision error of 0.48 g/L. The lactate model 210 demonstrated acceptable accuracy and precision with an RMSECV of 0.31 g/L, CV bias of -0.0174 g/L, and precision error of 0.31 g/L. Both models exhibited minimal bias and strong linearity, with R2 values of 0.94 for glucose and 0.92 for lactate, indicating their reliability for bioreactor process monitoring. Similarly, plot F in FIGS. 2G and 2H indicate the confidence measures of the glucose model 208 and the lactate model 210, respectively, to support evaluation of model performance and reliability, which may be considered as part of the selection criteria 298 in relation to the predicted glucose value 216 and the predicted lactate value 218 and, by extension, the carbon source value 220.

As previously noted, while the embodiments described herein utilize glucose and lactate as primary analytes for determining the carbon source value, it will be appreciated that additional analytes may be incorporated into the model system. For example, other metabolites or process variables may be included to further refine the composite control variable. Similarly, while certain embodiments utilize chemometric models such as PLS, alternative modeling approaches may be employed, including machine learning models capable of capturing nonlinear relationships within the spectral data.

### Cell Culture & Bioreactor Methods:

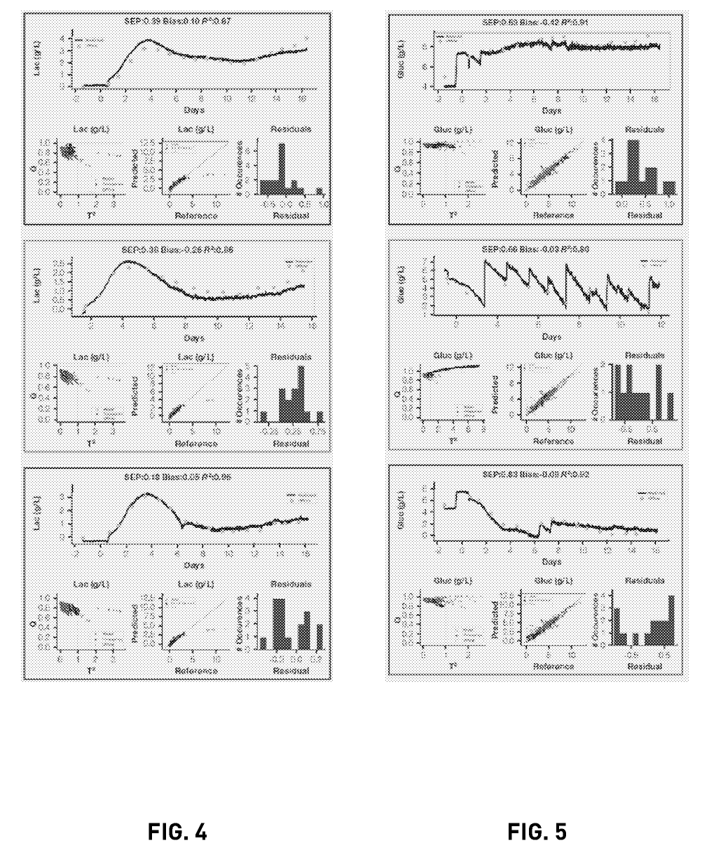

At least one experiment was performed in relation to at least one embodiment of the present disclosure. Such experiment is described below, and the results of such experiment are provided herein in FIG. 4 through FIG. 9.

miCHOTM CHO-Kl cells (ATUM) were inoculated in Efficient-ProTM medium (Gibco) supplemented with 1.5 mg/L insulin and 1 % anticlumping agent at a density of 0.75 million cells/mL in a 5 L glass bioreactor (Applikon). The bioreactor was operated at a temperature of 37°C, pH 7 ± 0.2, and dissolved oxygen (DO) maintained at 40%. The pH was controlled by the addition of CO2 for high pH and sodium carbonate for low pH adjustments. The cells were grown in standard fed-batch bioreactors, in duplicate, using a Thermo Fisher Scientific platform process with daily feeding starting day 3 with a volume specific feed rate of Efficient-Pro feed 3 and Enhancer (Gibco) fed either by bolus or continuous addition for a total run duration of 14 days. Daily samples were taken for Viable Cell Density (Beckman Coulter) and Metabolites (Nova Biomedical). Glucose additions were controlled using the conditions outlined in FIG. 3. The Thermo Scientific™ MarqMetrix™ All-In-One Process Raman Analyzer was used for the continuous control conditions. Real-time predictions of glucose and lactate were sent to the reactor control system (TruBio) where automated glucose supplementation was enabled using the logic outlined in FIG. 3.

FIGS. 4 and 5 depict glucose lactate prediction plot, respectively, with offline data. Three different glucose controls, discussed in FIG. 3, are being compared: (i) bolus standard control 604; (ii) automated continuous glucose control 602; and (iii) automated carbon source control 606, utilizing both glucose and lactate. Online predictions match closely with offline measurements.

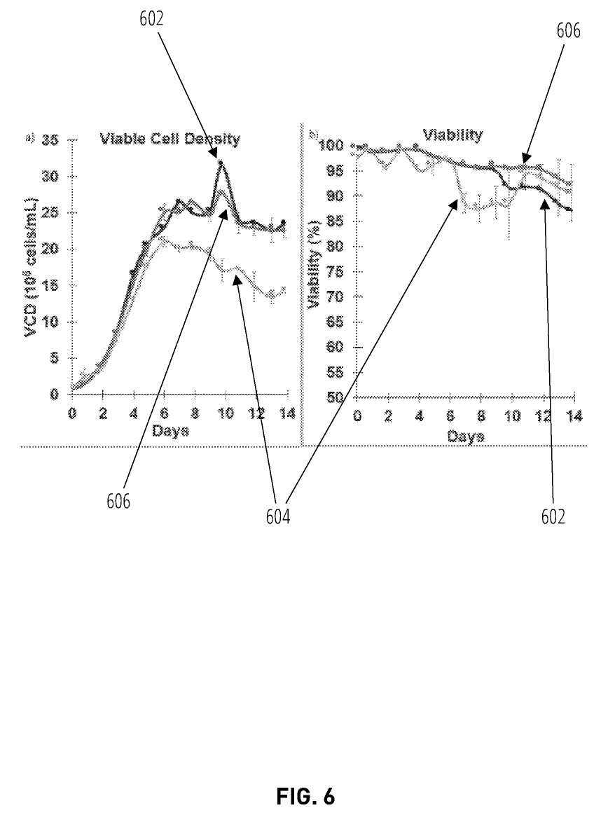

In FIG. 6, viable cell density, depicted in plot (a), and viability, depicted in plot (b), are compared for three different glucose controls: (i) bolus standard control 604; (ii) automated continuous glucose control 602; and (iii) automated carbon source control 606, utilizing both glucose and lactate. While some differences in viable cell density were observed, such differences are believed to be within historical averages.

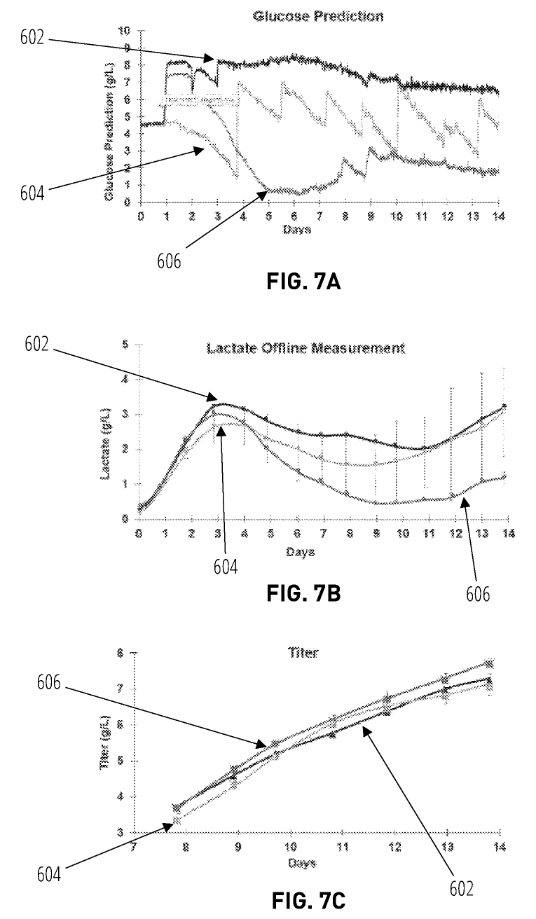

In FIG. 7A, glucose predictions from Raman spectral data 202 are depicted according to three different glucose controls: (i) bolus standard control 604; (ii) automated continuous glucose control 602; and (iii) automated carbon source control 606. Glucose levels were controlled at an overall lower level for the carbon source feedback control condition.

FIG. 7B depicts offline lactate measurements for three different glucose controls: (i) bolus standard control 604; (ii) automated continuous glucose control 602; and (iii) automated carbon source control 606. Lower levels of lactate accumulation, with lower variability, were achieved using automated carbon source control 606.

FIG. 7C depicts titer total accumulation for three different glucose controls: (i) bolus standard control 604; (ii) automated continuous glucose control 602; and (iii) automated carbon source control 606. Similar titers were achieved with ~ 10% higher titer when using automated carbon source control 606.

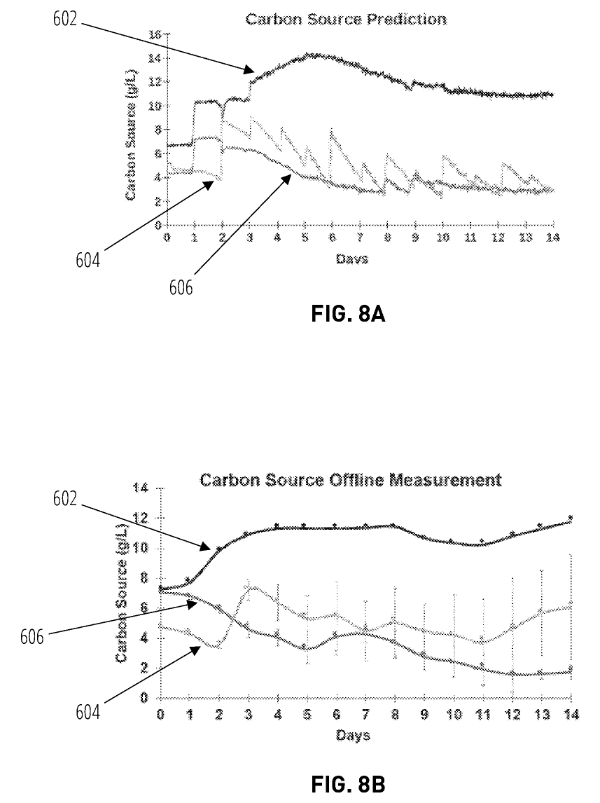

FIG. 8A shows total accumulation of carbon source in line prediction for three different glucose controls: Three different glucose controls are being compared: (i) bolus standard control 604; (ii) automated continuous glucose control 602; and (iii) automated carbon source control 606. Lower carbon source was maintained using carbon source feedback control.

FIG. 8B shows total offline accumulation of carbon source for three different glucose controls: (i) bolus standard control 604; (ii) automated continuous glucose control 602; and (iii) automated carbon source control 606. Offline measurements confirm lower levels of total carbon source for carbon source feedback control

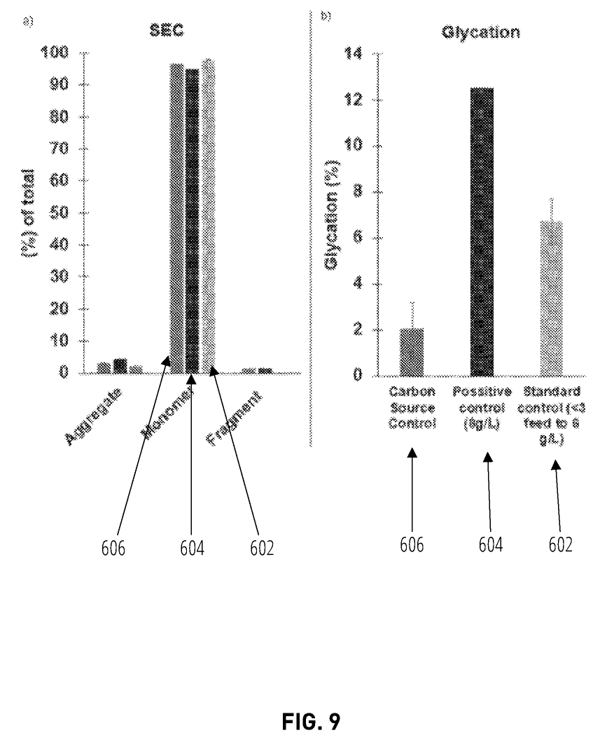

FIG. 9 shows protein quality quantification for SEC, in plot (a), and glycation, in plot (b), for three different glucose controls: (i) bolus standard control 604; (ii) automated continuous glucose control 602; and (iii) automated carbon source control 606. Similar SEC profiles were observed with decreasing amounts of glycation present with conditions controlled with lower average glucose concentrations with the lowest observed with the carbon source feedback control. As such, the proposed method showed reduced glycation of the mAb.

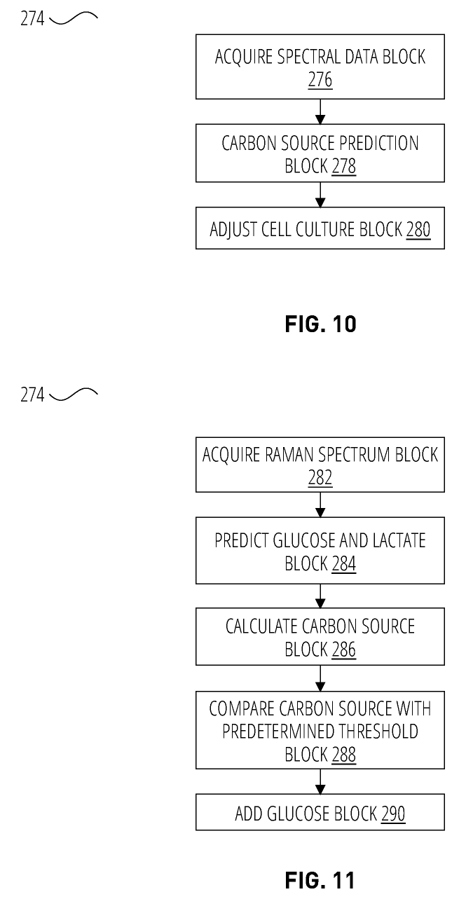

FIG. 10 depicts a control routine 274 in accordance with at least one embodiment of the present disclosure. Specifically: (a) in acquire spectral data block 276, spectral data 202 is acquired from the cell culture media 116; (b) in carbon source prediction block 278, a carbon source value 220 is predicted based on the spectral data 202 acquired from the cell culture media 116; and (c) in adjust cell culture block 280, the cell culture media 116 is adjusted responsive to the value of the carbon source value 220 in relation to the predetermined threshold 292.

FIG. 11 depicts a control routine 274 in accordance with at least one embodiment of the present disclosure. Specifically: (a) in acquire Raman spectrum block 282, Raman spectral data 202 is acquired from the cell culture media 116; (b) in predict glucose and lactate block 284, a predicted glucose value 216 and a predicted lactate value 218 are predicted based on the spectral data; (c) in calculate carbon source block 286, a carbon source value 220 is calculated based on the predicted glucose value 216 and predicted lactate value 218; (d) in compare carbon source with predetermined threshold block 288, the carbon source value 220 is compared with a predetermined threshold 292; and (e) in add glucose block 290, glucose is added to the cell culture media 116 in response to the carbon source value 220 in relation to the predetermined threshold 292.

Various embodiments of the present disclosure effectively provide a glucose control strategy which limits the recurrence of lactate accumulation toward the end of a standard fed batch culture using carbon source prediction generated from inline Raman spectra. The carbon source feedback control of glucose led to an increase in titer production of around 10% and decreased process variability, while also providing increased product quality and batch consistency.

What is claimed is:

1. A method for controlling a bioreactor, comprising:
acquiring spectral data from a cell culture media disposed within a bioreactor;
generating a predicted glucose value and a predicted lactate value;
determining a carbon source value by combining at least the predicted glucose value and the predicted lactate value;
comparing the carbon source value to a predetermined threshold; and
activating a pump system of the bioreactor to add a nutrient to the cell culture media according to the comparison.

2. The method of claim 1, wherein the carbon source value comprises a sum of the predicted glucose value and the predicted lactate value.

3. The method of claim 1, wherein the carbon source value comprises a multi-variable combination of at least two carbon energy sources present in the cell culture media.

4. The method of claim 1, wherein the pump system is activated in a binary manner to maintain a carbon energy source concentration within the cell culture media at the predetermined threshold.

5. The method of claim 1, wherein the pump system is activated to execute a bolus feeding schedule.

6. A method for controlling a bioreactor, comprising:
generating a carbon source value from spectral data acquired from a cell culture media disposed within a bioreactor according to a model system;
determining whether the carbon source value satisfies at least one selection criteria associated with the model system; and
controlling a pump system of the bioreactor according to the carbon source value and the at least one selection criteria.

7. The method of claim 6, wherein the model system is configured to generate the carbon source value from a glucose model and a lactate model, the glucose model configured to determine a predicted glucose value and the lactate model configured to determine a predicted lactate value.

8. The method of claim 7, wherein at least one of the glucose model and the lactate model is constructed using PLS, ANN, CNN, SVR, PCR, CLS, and LWR.

9. The method of claim 7, wherein at least one of the glucose model and the lactate model is trained according to historical data and processed data.

10. The method of claim 6, wherein the selection criteria is based on one or more characteristics of the model system, the one or more characteristics comprising feature importance metrics or model performance metrics.

11. The method of claim 10, wherein an alert component is activated if the carbon source value does not satisfy the at least one selection criteria.

12. The method of claim 6, wherein the model system is generated by aligning the spectral data with historical data based on temporal information.

13. The method of claim 12, wherein aligning the spectral data with historical data based on temporal information comprises:
continuously acquiring the spectral data at non-synchronized time points from the bioreactor;
associating the spectral data with timestamp data identifying the non-synchronized time points for the acquisition of the spectral data; and
aligning the spectral data with historical data according to the timestamp data.

14. A system for controlling a bioreactor, comprising:
a bioreactor comprising an interior chamber and a pump system, the interior chamber having a cell culture media disposed therein;
a probe configured to acquire spectral data from the cell culture media, the probe communicatively configured in connection with an analyzer for the collection of the spectral data;
a control system communicatively configured to receive the spectral data from the analyzer, the control system comprising:
a model system comprising a glucose model and a lactate model, the glucose model and the lactate model collectively configured to generate a carbon source value from the spectral data; and
a supervisory control component comprising a pump control component configured to control the pump system of the bioreactor to increase a carbon energy source concentration within the cell culture media.

15. The system of claim 14, wherein the supervisory control component further comprises a comparison component configured to compare the carbon source value with a predetermined threshold.

16. The system of claim 15, wherein the pump control component is configured to increase the carbon energy source concentration within the cell culture media according to the comparison component to maintain the carbon energy source concentration at the predetermined threshold.

17. The system of claim 14, wherein at least one of the glucose model and the lactate model is generated according to processed data, the processed data comprising historical data preprocessed using a Savitzky-Golay filter, a standard normal variate, and a mean centering.

18. The system of claim 14, wherein the model system further comprises a selection criteria, the model system configured to determine whether the carbon source value satisfies the selection criteria.

19. The system of claim 14, wherein the model system is configured to predict a future value for the carbon source value according to at least one machine learning model, the at least one machine learning model configured to use historical data.

20. The system of claim 14, wherein the probe comprises a Raman probe, the Raman probe itself comprising an optical system configured to acquire the spectral data.
